(19) United States
(12) Patent Application Publication
(10) Pub. No.: US 2007/0154439 A1
(43) Pub. Date: Jul. 5, 2007

(54) Cosmetic composition for use around the eyes

(76) Inventor: Paula Dorf, New York, NY (US)

Correspondence Address: Ward & Olivo, 708 Third Avenue, New York, NY, 10017, US

(21) Appl. No.: 11/325,253

(22) Filed: Jan. 4, 2006

(51) Int. Cl. A61K 8/97; A61K 8/37

(52) U.S. Cl. 424/70.31; 424/74

(57) ABSTRACT

A cosmetic composition is provided that is particularly intended for application to areas surrounding the eyes. The composition contains skin conditioning emollients, skin conditioning occlusives, moisturizers, a rich source of vitamin E, dipalmitoyl hydroxyproline, botanical extracts, antioxidants, and preservatives specifically designed to soften, nourish, and moisturize the skin around the eyes. In addition, the cosmetic may be used to reduce the appearance of wrinkles, cracks and/or crevices that surround the eye which are also a result of the aging process. The cosmetic composition also reduces puffiness around the eyes which often appear as the day progresses.

### FIELD OF THE INVENTION

The present invention relates to a cosmetic composition for application to the area of skin surrounding the eyes. More specifically, the invention relates to a composition intended for topical application to the fine facial lines that form around the eyes during the aging process. The composition is designed to penetrate the skin and reduce the appearance of wrinkles by simulating the contraction of collagen fibers under the skin. In addition, the composition contains moisturizers, occlusives and vitamins to soften and nourish the skin surrounding the eyes.

### BACKGROUND OF THE INVENTION

Facial lines and wrinkles are particularly noticeable and occur most frequently around the eye area. Reduction of, or at least covering, the appearance of facial lines and/or wrinkles is an important function of a cosmetic skin composition. The present invention is a composition for a cosmetic for application to skin surfaces, such as the facial lines on or around the eye area.

The appearance of fine facial lines and wrinkles is a serious concern for many during the aging process. While most people would prefer that such lines and wrinkles be completely eliminated, this is impractical. However, steps can be taken to reduce their prominence on the face.

External or internal factors can both lead to the emergence of symptoms of aging. Moreover, as skin ages, the synthesis of collagen or other macromolecules in connective tissue is slowed; proteolysis, induced by solar radiation, is accelerated and the skin grows thinner and loses elasticity.

Numerous cosmetic compositions intended to improve the appearance of facial skin have been proposed to date. These include moisturizing products, anti-wrinkle creams, smoothing and soothing lotions. Frequently, however, those products have side effects, are associated with stability problems and/or do not make good their promise over time. This is, in particular, the case for formulae containing vitamins and plant extracts.

The invention described herein discloses an improved cosmetic composition which is specifically designed to soften, nourish, and moisturize the skin around the eyes. In addition, the cosmetic may be used to reduce the appearance of wrinkles, cracks and/or crevices that surround the eye which are also a result of the aging process. The cosmetic composition also reduces puffiness around the eyes.

### SUMMARY OF THE INVENTION

The present invention provides a novel cosmetic composition that is particularly intended for application to areas surrounding the eyes. The composition contains skin conditioning emollients, skin conditioning occlusives, moisturizers, a rich source of vitamin E, dipalmitoyl hydroxyproline, botanical extracts, antioxidants, and preservatives specifically designed to soften, nourish, and moisturize the skin around the eyes. In addition, the cosmetic may be used to reduce the appearance of wrinkles, cracks and/or crevices that surround the eye which are also a result of the aging process.

The cosmetic composition also may contain chemicals designed to reduce puffiness around the eyes which often appears as the day progresses, as well as soften and smooth the skin in order to minimize the appearance of facial lines.

In light of the above described disadvantages and insufficiencies of the prior art, it is an object of the invention herein to provide a cosmetic composition for application to the area surrounding the eyes to soften, nourish, and moisturize the skin around the eyes.

It is another object of the invention to provide a cosmetic composition for application to the skin surrounding the eyes such that the application of the cosmetic composition may be smooth, seamless, and long-lasting.

Still another object of the invention is to provide a product that is appropriate for use around the eyes.

Yet another object of the invention to provide a product that imparts a desirable feel on the skin.

Yet another object of the invention is to provide a product that is multi-purpose and may be used for eyeliner, eye shadow, and other cosmetics.

Another object of the present invention is to create a cosmetic composition which reduces the appearance of puffiness around the eyes.

Yet another object of the present invention is to create a cosmetic composition which visibly reduces the appearance of wrinkles.

Still a further object of the present invention is to provide a composition which simulates the contraction of collagen fibers.

Other objects, features, and characteristics of the present invention, as well as the methods of operation and functions of the related elements of the structure, and the combination of parts and economies of manufacture, will become more apparent upon consideration of the following detailed description with reference to the accompanying drawings, all of which form a part of this specification.

### DETAILED DESCRIPTION OF THE PREFERRED EMBODIMENT

A detailed illustrative embodiment of the present invention is disclosed herein. However, techniques, systems and operating structures in accordance with the present invention may be embodied in a wide variety of forms and modes, some of which may be quite different form those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Moreover, well known methods, procedures, and substances for both carrying out the objectives of the present invention and illustrating the preferred embodiment are incorporated herein but have not been described in detail as not to unnecessarily obscure aspects of the present invention.

As discussed above, the present invention relates generally to a product to diminish the effect of facial lines and wrinkles, especially for the areas on or surrounding the eyes.

A preferred embodiment of the present invention comprises a composition made from cyclomethicone, paraffin wax, polybutene, bis-diglyceryl polyacyladipate-2, raspberry seed oil, ethylhexyl ethylhexanoate, jojoba oil, c10-c30 cholesterol/lanoesterol esters, chamomile extract, dipalmitoyl hydroxyproline, methylparaben, propylparaben, PEG-8, sorbitan isostearate, tocopherol, ascorbyl palmitate, ascorbic acid, and citric acid.

The present invention utilizes cyclomethicone, jojoba oil, bis-diglyceryl polyacetadipate-2 as skin conditioning emollients. These emollients help reduce skin flaking as well as impart a soft, smooth appearance to skin. Of course other emollients may be substituted as is known in the art.

The invention further comprises a rich source of vitamin E as an anti-inflammatory compound essential for the prevention of rash, eczema, and other skin lesions. The vitamin E also serves as an antioxidant for the skin, thereby keeping the user's skin young looking and vibrant.

It is contemplated that alternative embodiments utilize a variety of other vitamins, including vitamin A, vitamin C, vitamin D, any complex of vitamin B, and vitamin K.

The chemical composition of the present invention allows for smooth application to the skin around the eyes. The composition is viscous when applied to the facial area and deposits a transparent material in and around facial lines, thereby preventing other makeup from entering said facial lines and highlighting their presence. Some acceptable viscosity increasing agents which may be used with the invention include alkyl galactomannan, silica, talc, magnesium silicate, sorbitol, colloidal silicone dioxide, magnesium aluminum silicate, zinc stearate, wool wax alcohol, sorbiton, sesquioleate, cetyl hydroxy ethyl cellulose and other modified celluloses. Some suitable solvents include propylene glycol, glycerine, cyclomethicone, polyethylene glycols, hexalene glycol, diol and multi-hydroxy based solvents.

In an alternative embodiment of the invention, the composition also comprises a masking oil. Suitable masking oils include, for example, polydecene, polybutene, PPG-14 butyl ether, non-volatile silicones, isopropyl myristate, isopropyl palmitate, C₁₂-C₁₅ alkyl benzoates, and mineral oils.

The components used in the composition comprise at least one skin conditioning occlusive which increases the water content of the skin, thereby protecting dry or damaged skin. In the preferred embodiment of the present invention, the skin conditioning occlusive is paraffin wax because it also acts as a binder. Alternative embodiments utilize other known skin occlusives, well known in the art.

The compositions used in the method of the invention contain an oil to permeate and penetrate the skin, providing a moisturizer. The oils can lubricate and soften dry skin as they provide a therapeutic relaxant to tight wrinkled skin. In the preferred embodiment, the composition utilizes polybutene as a moisturizing oil.

In alternative embodiments, several nonvolatile oils, volatile oils, and mixtures thereof can be added.

Nonvolatile oils that may be used include such esters as isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, and fatty alcohols such as oleyl alcohol, isocetyl alcohol, and the like.

In another alternative embodiment, the oil may also comprise naturally occurring glyceryl esters of fatty acids, or triglycerides. Both vegetable and animal sources may be used. Examples of such oils include castor oil, lanolin oil, triisocetyl citrate, C.sub.10-18 triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

In yet another alternative embodiment, synthetic or semi-synthetic glyceryl esters can be substituted for oils. For example, fatty acid mono-, di-, and triglycerides which are natural fats or oils that have been modified may be substituted for oils. These oil substitutes include acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, and so on.

Also suitable for use as the oil are nonvolatile hydrocarbons such as isoparaffins, hydrogenated polyisobutene, mineral oil, squalene, petrolatum, and so on.

As previously stated, the composition contains paraffin wax to thicken or bind the composition. The wax thickens and stiffens the composition and can possess moisturizing qualities. Alternative embodiments of the invention can include animal waxes, plant waxes, mineral waxes, silicone waxes, synthetic waxes, and petroleum waxes.

Examples of waxes that can be used include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, stearoxy dimethicone, dimethicone behenate, stearyl dimethicone, and the like, as well synthetic homo- and copolymer waxes from the ethylene series. Preferred waxes are paraffins.

Additional ingredients in the composition include antioxidants, UV absorbing compounds and other materials described herein.

The antioxidants which may be used in the formulations are PEG-8, butylated hydroxyanisole, butylated hydroxytoluene, ascorbic acid, citric acid, tocopherol, tocopheryl acetate, ascorbyl palmitate, retinol, retinyl palmitate, hydroquinone and proanthocyanadines. The preferred composition utilizes PEG-8, tocopherol, ascorbyl palmitate, and citric acid and antioxidants.

The ultra-violet absorbing compounds which may be used in the formulations are selected from the group consisting of octyl methoxycinnimmate, p-aminobenzoic acid, p-aminobenzoic acid esters, benzophenone-3 and other FDA approved sunscreens.

In addition, the composition comprises at least one preservative. Preferred preservatives include methylparaben and propylparaben, however, other known preservatives can be utilized in accordance with the present invention.

The cosmetic composition of the present invention utilizes one or more botanical extracts. The extract acts to sooth and repair damaged skin. In the preferred embodiment, the botanical extract is chamomile extract, however, any other known extract can be utilized in accordance with the present invention.

The present invention utilizes dipalmitoyl hydroxyproline, which rehydrates the skin by improving the ceramide activity in the skin and assists in the prevention of DNA damage.

In addition, it utilizes the synergistic effect of palmitoyl hexapeptide and ceramide 2 to combat the signs of aging and reduce the appearance of fine lines and wrinkles. Palmitoyl hexapeptide stimulates the dermal matrix to produce collagen and elastin in the skin which helps “plump” up the skin thereby reducing the depth of wrinkles and fine lines and giving the skin a smoother appearance. Ceramide 2 strengthens and repairs the skin's barrier function to enhance the skin's natural hydration system. Dipalmitoyl hydroxyproline also improves cell cohesion, enhances water retention and reduces skin dryness.

The constituent compounds of the present invention can be combined in a well known manner to create a cream, lotion, or other cosmetically acceptable composition. The following is a preferred composition of the present invention:

### EXAMPLE COSMETIC COMPOSITION

| Ingredient | Weight Percentage |
|---|---|
| Cyclomethicone | 32.00 |
| Paraffin Wax | 23.00 |
| Polybutene | 10.00 |
| Bis-diglyceryl polyacyladipate-2 | 8.00 |
| Rubus Idaeus (raspberry) seed oil | 7.00 |
| Ethylhexyl ethylhexanoate | 6.50 |
| Jojoba oil | 6.00 |
| C10-c30 cholesterol/lanoesterol esters | 5.20 |
| Chamomile extract | 1.00 |
| Dipalmitoyl hydroxyproline | 1.00 |
| Methylparaben | 0.100 |
| Propylparaben | 0.100 |
| PEG-8 | 0.049 |
| Sorbitan isostearate | 0.020 |
| Tocopherol | 0.024 |
| Ascorbyl palmitate | 0.005 |
| Ascorbic acid | 0.001 |
| Citric acid | 0.001 |

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof.

What is claimed is:

1. A chemical composition for a cosmetic, wherein said composition comprises:
at least one skin conditioning emollient;
at least one skin conditioning occlusive;
at least one moisturizer;
a source of vitamin E;
dipalmitoyl hydroxyproline;
at least one botanical extract;
at least one antioxidant; and
at least one preservative.

2. A chemical composition according to claim 1, wherein said skin conditioning emollient comprises at least one selected from the group consisting of cyclomethicone, jojoba oil, bis-diglyceryl polyacyladipate-2, ethylhexyl ethyl hexanoate, and combinations thereof.

3. A chemical composition according to claim 1, wherein said skin conditioning occlusive comprises paraffin wax.

4. A chemical composition according to claim 1, wherein said skin conditioning occlusive comprises at least one selected from the group consisting of bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, stearoxy dimethicone, dimethicone behenate, stearyl dimethicone, and synthetic homo- and copolymer waxes from the ethylene series.

5. A chemical composition according to claim 1, wherein said moisturizer comprises polybutene, paraffin wax, or mixtures thereof.

6. A chemical composition according to claim 1, wherein said moisturizer comprises at least one selected from the group consisting of ascorbyl palmitate, dioctyl malate, isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, oleyl alcohol, and isocetyl alcohol, naturally occurring glyceryl esters of fatty acids, or triglycerides, castor oil, lanolin oil, triisocetyl citrate, C.sub.10-18 triglycerides, caprylic/capric/triglycerides, coconut oil, corn oil, cottonseed oil, linseed oil, mink oil, olive oil, palm oil, illipe butter, rapeseed oil, soybean oil, sunflower seed oil and walnut oil.

7. A chemical composition according to claim 1, wherein said vitamin E source comprises raspberry seed oil.

8. A chemical composition according to claim 1, wherein said botanical extract comprises chamomile extract.

9. A chemical composition according to claim 1, wherein said antioxidant comprises at least one selected from the group consisting of PEG-8, tocopherol, ascorbyl palmitate, ascorbic acid, and citric acid.

10. A chemical composition according to claim 1, wherein said preservative comprises methylparaben, propylparaben, or combinations thereof.

11. A chemical composition according to claim 1, wherein said preservative comprises at least one selected from the group consisting of butylated hydroxyanisole, butylated hydroxytoluene, tocopherol, tocopheryl acetate, ascorbyl palmitate, retinol, retinyl palmitate, hydroquinone and proanthocyanadines.

12. A chemical composition according to claim 1 further comprising an ultra-violet absorbing compound.

13. A chemical composition according to claim 12, wherein said ultra-violet absorbing compound is at least one selected from the group consisting of octyl methoxycimmate, p-aminobenzoic acid, p-aminobenzoic acid esters, benzophenone-3 and other FDA approved sunscreens.

14. A chemical composition according to claim 1 further comprising a viscosity increasing agent.

15. A chemical composition according to claim 14, wherein said viscosity increasing agent is at least one selected from the group consisting of alkyl galactomannan, silica, talc, magnesium silicate, sorbitol, colloidal silicone dioxide, magnesium aluminum silicate, zinc stearate, wool wax alcohol, sorbiton, sesquioleate, cetyl hydroxy ethyl cellulose, sorbitan isostearate, and mixtures thereof.

16. A chemical composition according to claim 1 further comprising vitamin A.

17. A chemical composition according to claim 1, wherein said composition further comprises synthetic or semi-synthetic glyceryl esters.

18. A chemical composition according to claim 4, wherein said synthetic or semi-synthetic glyceral esters are selected from the group consisting of: acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, and so on.

19. A chemical composition according to claim 1, wherein said composition comprises nonvolatile hydrocarbons.

20. A chemical composition according to claim 19, wherein said nonvolatile hydrocarbons are selected from the group consisting of: isoparaffins, hydrogenated polyisobutene, mineral oil, squalene, and petrolatum.

21. A cosmetic composition comprising:
cyclomethicone;
paraffin wax;
polybutene;
bis-diglyceryl polyacyladipate-2;
raspberry seed oil;
ethylhexyl ethylhexanoate;
jojoba oil;
c10-c30 cholesterol/lanoesterol esters;
chamomile extract;
dipalmitoyl hydroxyproline;
methylparaben;
propylparaben;
PEG-8;
sorbitan isostearate;
tocopherol;
ascorbyl palmitate;
ascorbic acid; and
citric acid.

22. A cosmetic composition comprising by weight:
32% cyclomethicone;
23% paraffin wax;
10% polybutene;
8% bis-diglyceryl polyacyladipate-2;
7% raspberry seed oil;
6.5% ethylhexyl ethylhexanoate;
6% jojoba oil;
5.2% C10-C30 cholesterol/lanoesterol esters;
1% chamomile extract;
1% dipalmitoyl hydroxyproline;
0.1% methylparaben;
0.1% propylparaben;
0.049% PEG-8;
0.020% sorbitan isostearate;
0.024% tocopherol;
0.005% ascorbyl palmitate;
0.001% ascorbic acid; and
0.001% citric acid.
